(19) United States
(12) Patent Application Publication
(10) Pub. No.: US 2017/0304835 A1
(43) Pub. Date: Oct. 26, 2017

(54) DRY ICE FREEZE-PULVERIZER

(71) Applicants: AiSTI SCIENCE CO., Ltd., Wakayama (JP); Aichi Science & Technology Foundation, Aichi (JP)

(72) Inventors: Isao SAITO, Aichi (JP); Ryoichi SASANO, Wakayama (JP); Kenji KONISHI, Wakayama (JP)

(21) Appl. No.: 15/529,531

(22) PCT Filed: Oct. 15, 2015
(86) PCT No.: PCT/JP2015/079137

§ 371 Date: May 25, 2017

(30) Foreign Application Priority Data
Nov. 26, 2014 (JP) 2014-238317

(51) Int. Cl. B02C 17/14; C12M 1/33; G01N 1/28; C12M 3/08; B02C 19/00; C12M 1/00

(52) U.S. Cl. CPC B02C 17/14; G01N 1/286; C12M 3/08; C12M 1/33; C12M 1/00; B02C 19/00

(57) ABSTRACT

A dry ice freeze-pulverizer includes pulverizing container and pulverizer main body. The pulverizing container includes a container main body that includes a bottom plate and a side plate erected from a peripheral edge of the bottom plate, has an upper surface opening, and is provided with a pulverizing blade therein, and a lid that closes the upper surface opening and has a vent hole. The pulverizer main body includes a drive device configured to rotate the pulverizing blade. The side plate of the container main body is formed with a vacuum double structure that is obtained by evacuating an internal space formed by opposing metal plates.

### TECHNICAL FIELD

The present invention relates to a dry ice freeze-pulverizer that pulverizes an analytical sample in the physical and chemical analysis fields, the food field, or the like, in a state where the sample is frozen by adding dry ice to the sample.

### BACKGROUND ART

On extraction for pesticide residues in food by sampling a small amount of a sample, it is particularly important to uniformize a pulverized sample, and dry ice freeze-pulverization in which a sample is pulverized in a state where the sample is frozen by adding dry ice to the sample is known as a very useful pulverizing method to be carried out when a sample is pulverized at normal temperature with a food cutter, a food processor, a blender, or the like (see, for example, Non-Patent Literature 1 to 3).

For example, in dry ice freeze-pulverization of Non-Patent Literature 3, after snow-like dry ice is added to a shredded sample and the mixture is mixed well to precool the sample, the sample is put into a precooled pulverizing container, and is freeze-pulverized by rotating a pulverizing blade within the pulverizing container by a drive device of a pulverizer such as a food processor or a blender.

### CITATION LIST

### Non Patent Literature

[NPL 1] Isao Saito, et al., “Dorai aisu tenka kinshitsuka sousa wo kuwaeta shokuhinchu zanryu nouyaku issei bunseki (Simultaneous analysis of pesticide residue in food using dry ice adding/homogenizing operation)”, the proceedings of the 98th annual meeting of Japanese Society for Food Hygiene and Safety, Oct. 8, 2009, p.49

[NPL 2] Ryoichi Sasano, et al., “Shouryou shiryou sanpuringu notameno dorai aisu touketsu hunsai no kentou (Examination of dry ice freeze-pulverization for sampling small amount of sample)”, the proceedings of the 106th annual meeting of Japanese Society for Food Hygiene and Safety, Nov. 21, 2013, p. 115

[NPL 3] Product information of “precooling type dry ice freeze-pulverizing set”, AiSTI SCIENCE CO., LTD., [searched on Nov. 12, 2014], the internet <URL: http://www.aisti.co.jp/product/dryice₁₃ set>

### SUMMARY OF THE INVENTION

### Problems to be Solved by the Invention

As described above, when a sample is pulverized by a pulverizer in a state where the sample is frozen by dry ice, analysis time can be shortened since time and effort to freeze the sample by refrigeration can be omitted due to the use of dry ice. In addition, a uniformized powdery sample can be obtained.

However, with the configuration of such a dry ice freeze-pulverizer, in pulverizing a sample at normal temperature, the temperature of the inner surface of a side plate of the pulverizing container may increase due to influence of the ambient temperature, and thus part of the frozen pulverized sample may melt and freeze again at the inner surface of the side plate of the pulverizing container to form lumps and adhere to the inner surface of the side plate. In this case, since a part of the pulverized sample does not reach the pulverizing blade in the lower part of the pulverizing container, and forms lumps and adheres to the inner surface of the side plate, the degree of pulverization of the sample decreases.

In this situation, in order to prevent a sample from adhering to the inner surface of the side plate of the pulverizing container as described above, it is conceivable to cover the periphery of the side plate of the pulverizing container with a heat insulator (see Non-Patent Literature 3), and such a configuration is effective for enhancing heat insulation properties.

However, it is difficult to cover the entirety of the side plate in the configuration with the heat insulator. In addition, depending on the usage conditions, required heat insulation properties are not achieved merely by using such a heat insulator, and a pulverized sample may form lumps and adhere to the inner surface of the side plate.

Moreover, in the configuration in which the periphery of the side plate of the pulverizing container is covered with the heat insulator, it is necessary to remove the heat insulator when washing the container. Thus, work to remove and mount the heat insulator is cumbersome, and it also becomes necessary to wash the heat insulator since the heat insulator is prone to become soiled.

Furthermore, since a large amount of carbon dioxide gas is generated by sublimation of dry ice during dry ice freeze-pulverization, it is necessary to form a vent hole for releasing the gas, in the lid of the pulverizing container. Thus, a part of the frozen pulverized sample may spill out of the container through the vent hole together with the gas.

Therefore, the conventional dry ice freeze-pulverizer is suitable for obtaining a uniformized powdery sample, but has room for improvement from the standpoint of further enhancing the heat insulation properties to inhibit a sample from adhering to the inner surface of the side plate and from the standpoint of inhibiting a frozen pulverized sample from spilling out of the container, in order to more efficiently obtain a uniformized pulverized sample.

Therefore, in view of the above-described circumstances, an object of the present invention is to provide a thy ice freeze-pulverizer that allows a uniformized pulverized sample to be more efficiently obtained.

### Solution to the Problems

In order to attain the above object, a dry ice freeze-pulverizer according to the present invention is a dry ice freeze-pulverizer including: a pulverizing container including a container main body that includes a bottom plate and a side plate erected from a peripheral edge of the bottom plate, has an upper surface opening, and is provided with a pulverizing blade therein, and a lid that closes the upper surface opening and has a vent hole; and a pulverizer main body including a drive device configured to rotate the pulverizing blade, wherein the side plate of the container main body is formed with a vacuum double structure that is obtained by evacuating an internal space formed by opposing metal plates.

With such a configuration, since the side plate of the container main body has the vacuum double structure, heat conduction and heat transmission by convection can be significantly reduced.

Therefore, since the heat insulation properties of the container structure of the container main body are very high, elevation in temperature of the inner surface of the side plate of the container main body due to influence of the ambient temperature can be inhibited during pulverization of a sample at normal temperature. Thus, part of a frozen pulverized sample can be inhibited from melting and freezing again at the inner surface of the side plate to form lumps and adhere to the inner surface of the side plate. Accordingly, the uniformity of the pulverized sample improves and a uniformized pulverized sample can be more efficiently obtained, and also wasteful consumption of dry ice can be prevented.

Moreover, as compared to a configuration in which the periphery of the side plate of the pulverizing container is covered with a heat insulator, work to remove the heat insulator in washing the container and the heat insulator, and work to mount the heat insulator after washing, are unnecessary, so that the maintenance workability is improved.

Here, one or each of the bottom plate and the lid is preferably formed with a vacuum double structure that is obtained by evacuating an internal space formed by opposing metal plates.

With such a configuration, the heat insulation properties of the pulverizing container are further enhanced, thereby increasing the inhibiting effect on elevation in temperature of the inner surface of the side plate of the container main body due to the ambient temperature during pulverization of a sample at normal temperature. Thus, the effect of inhibiting part of a frozen sample being pulverized from forming lumps and adhering to the inner surface of the side plate, is further enhanced. Accordingly, the uniformity of the pulverized sample further improves and a uniformized pulverized sample can be further efficiently obtained, and also the effect of preventing wasteful consumption of dry ice is further enhanced.

In addition, more suitably, a radiant heat transfer prevention body that serves as a mirror is provided in the internal space.

With such a configuration, since the radiant heat transfer prevention body, which serves as a mirror, is provided in the internal space, heat transmission by radiation can also be significantly reduced, so that the heat insulation properties of the pulverizing container are still further enhanced.

Therefore, the effect of inhibiting the temperature of the inner surface of the side plate of the container main body from rising due to influence of the ambient temperature during pulverization of a sample at normal temperature, is still further enhanced. Thus, the effect of inhibiting part of a frozen pulverized sample from forming lumps and adhering to the inner surface of the side plate, is still further enhanced. Accordingly, the uniformity of the pulverized sample still further improves, and a uniformized pulverized sample can be further efficiently obtained, and also the effect of preventing wasteful consumption of dry ice is still further enhanced.

Furthermore, a filter member that prevents a sample within the pulverizing container from passing therethrough while allowing gas to pass therethrough is preferably provided in the vent hole of the lid.

With such a configuration, since the filter member prevents a sample from passing therethrough while allowing gas to pass therethrough, a sample does not bubble over while maintaining the function to discharge carbon dioxide gas generated in a large amount when dry ice sublimates during dry ice freeze-pulverization. Thus, a uniformized pulverized sample can be still further efficiently obtained, and also contamination of the surroundings can be prevented.

### Advantageous Effects of the Invention

As described above, the dry ice freeze-pulverizer according to the present invention achieves significant effects such as the following effects.

(1) Since the heat insulation properties of the container main body are very high, a rise in the temperature of the inner surface of the side plate of the container main body due to influence of the ambient temperature during pulverization of a sample at normal temperature can be inhibited. Thus, part of a frozen sample being pulverized can be inhibited from melting and freezing again at the inner surface of the side plate to form lumps and adhere to the inner surface of the side plate. Accordingly, the uniformity of the pulverized sample improves and a uniformized pulverized sample can be more efficiently obtained, and also wasteful consumption of dry ice can be prevented.

(2) As compared to a configuration in which the periphery of the side plate of the pulverizing container is covered with a heat insulator, work to remove the heat insulator in washing the container and the heat insulator and work to mount the heat insulator after washing are unnecessary, so that the maintenance workability is enhanced.

(3) With the configuration in which the filter member is provided in the vent hole of the lid, a sample does not bubble over while maintaining the function to discharge carbon dioxide gas generated in a large amount when dry ice sublimates. Thus, a uniformized pulverized sample can be still further efficiently obtained, and also contamination of the surroundings can be prevented.

### BRIEF DESCRIPTION OF THE DRAWINGS

FIG. 1 is a front view of a dry ice freeze-pulverizer according to an embodiment of the present invention.

FIG. 2 is a partially cross-sectional right side view of the dry ice freeze-pulverizer.

FIG. 3 is an exploded cross-sectional front view of a lid, a filter member, and a filter holder.

FIG. 4 is a main part enlarged cross-sectional front view showing an example where the lid is formed with a vacuum insulation double structure.

### DESCRIPTION OF EMBODIMENTS

Next, embodiments of the present invention will be described in detail with reference to the accompanying drawings. The present invention is not limited to the embodiments shown in the accompanying drawings and includes all embodiments that meet the requirements described in the claims.

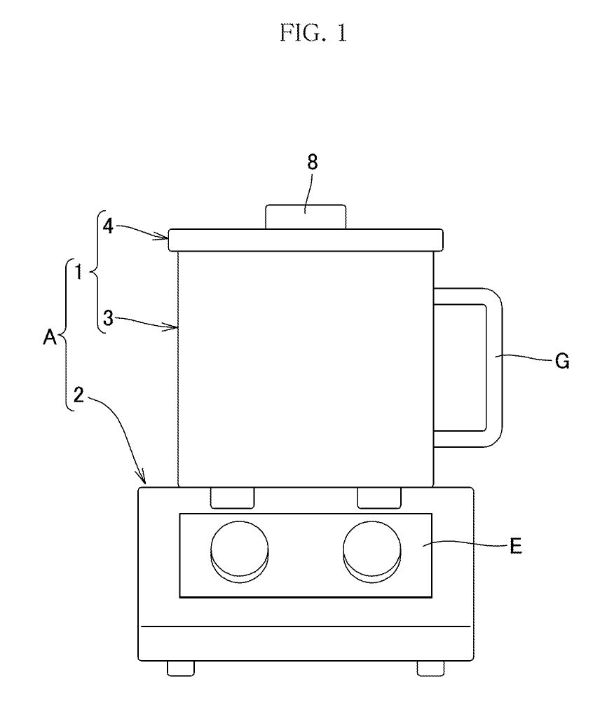

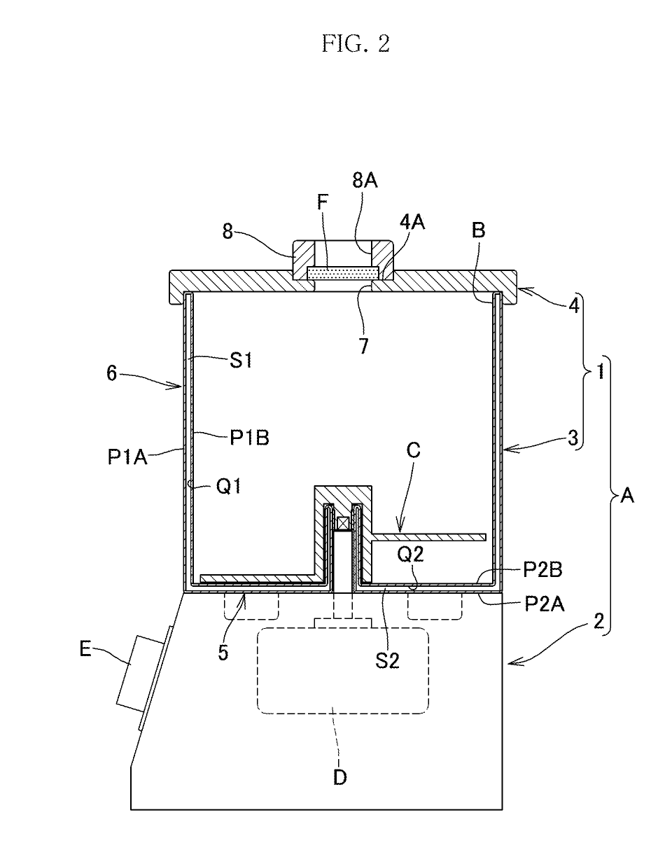

As shown in a front view of FIG. 1 and a partially cross-sectional right side view of FIG. 2, a dry ice freeze-pulverizer A according to an embodiment of the present invention includes: a pulverizing container 1 in which a sample to be freeze-pulverized with dry ice is to be contained; and a pulverizer main body 2 including a power unit in which a drive device D including, for example, an electric motor and a speed reducer is driven by operating an operation panel E in a state where the sample is put in the pulverizing container 1, thereby to rotate a pulverizing blade C within the pulverizing container 1.

Here, the pulverizing container 1 can be removed from the pulverizer main body 2 and carried, for example, by holding a grip G. In addition, in a state where the pulverizing container I is placed at a predetermined position on the pulverizer main body 2, an output shaft of the drive device D is connected to the pulverizing blade C within the pulverizing container 1, so that a drive torque of the drive device D is transmitted to the pulverizing blade C.

The pulverizing container 1 includes: a container main body 3 that includes a bottom plate 5 and a side plate 6 erected from the peripheral edge of the bottom plate 5, has an upper surface opening B, and is provided with the pulverizing blade C therein; and a lid 4 that closes the upper surface opening B and has a vent hole 7 formed therein.

The side plate 6 of the container main body 3 is fowled with a vacuum double structure that is obtained by evacuating an internal space S1 formed by opposing metal plates P1A and P1B that are, for example, stainless steel plates, and a radiant heat transfer prevention body Q1 that servers as a mirror and is formed from, for example, copper foil is provided on the outer surface (vacuum-side surface) of the metal plate P1B of the internal space S1.

In addition, the bottom plate 5 of the container main body 3 is formed with a vacuum double structure that is obtained by evacuating an internal space S2 formed by opposing metal plates P2A and P2B that are, for example, stainless steel plates, and a radiant heat transfer prevention body Q2 that servers as a mirror and is formed from, for example, copper foil is provided on the outer surface (vacuum-side surface) of the metal plate P2B of the internal space S2.

Here, the internal spaces S1 and S2 are connected to each other, and thus are evacuated together.

As shown in the partially cross-sectional right side view of FIG. 2, a disposable filter member F that prevents a sample within the pulverizing container 1 from passing therethrough while allowing gas to pass therethrough and is formed from, for example, cotton, a fabric, or the like, is provided in the vent hole 7 formed in a center portion of the lid 4.

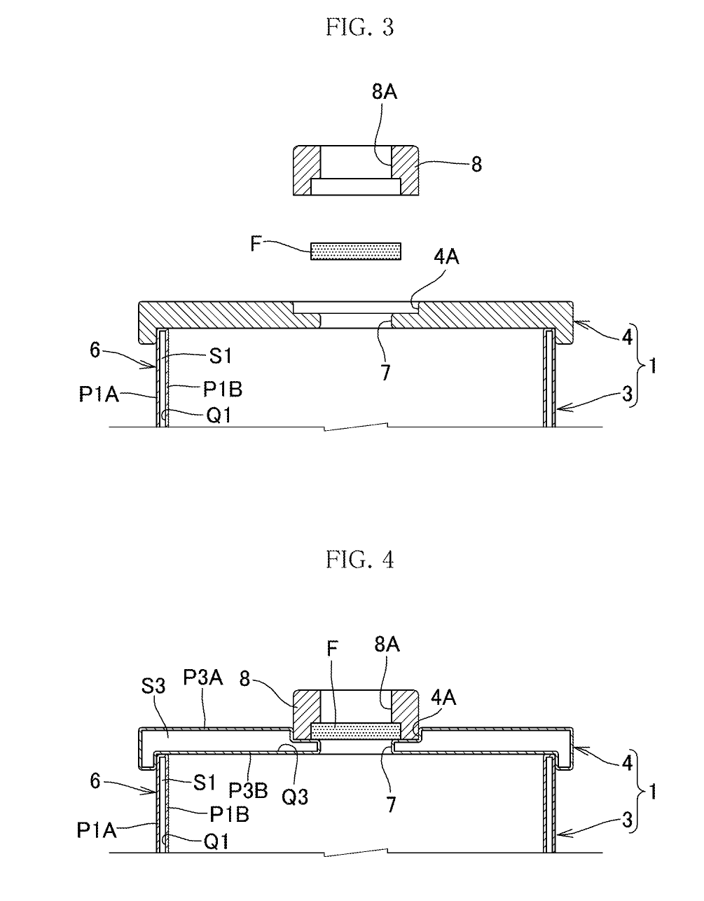

Here, the filter member F is held by a filter holder 8 in a state where the filter member F is placed in a recess 4A on the upper surface of the lid 4. Thus, the filter member F can easily be replaced by removing the filter holder 8 as shown in an exploded cross-sectional front view of FIG. 3.

Next, an example of dry ice freeze-pulverization by the pulverizer A will be described.

(1) First, a sample (at normal temperature or kept cold) is shredded and put into a precooling container.

(2) When dry ice to be used is in the form of a plate or in the form of pellets, the dry ice is put into the pulverizing container 1 and pulverized by the pulverizer A. The vaporized dry ice is discharged through the vent hole 7 of the lid 4, the filter member F, and a through hole 8A of the filter holder 8.

(3) Next, the dry ice pulverized in (2) is sprinkled over the sample within the precooling container in (1) approximately in the same amount as the sample, and the sample is precooled by shaking the precooling container in a state where a lid of the container is slightly opened.

(4) Next, the pulverizing container 1 is cooled by putting a small amount of dry ice into the pulverizing container 1 and pulverizing the dry ice in the pulverizer A.

(5) Next, a uniformized powdery sample is obtained by putting the sample precooled in (3) into the pulverizing container 1 and pulverizing the sample by the pulverizer A. The vaporized dry ice is discharged through the vent hole 7 of the lid 4, the filter member F, and the through hole 8A of the filter holder 8. In addition, because of presence of the filter member F, the sample does not bubble over.

With the configuration of the dry ice freeze-pulverizer A described above, since the side plate 6 and the bottom plate 5 of the container main body 3 have the vacuum double structure, heat conduction and heat transmission by convection can be significantly reduced. In addition, since the radiant heat transfer prevention bodies Q1 and Q2, which serve as mirrors, are provided in the internal spaces S1 and S2 of the side plate 6 and the bottom plate 5, heat transmission by radiation can also be significantly reduced.

Therefore, since the heat insulation properties of the container main body 3 are very high, a rise in the temperature of the inner surface of the side plate 6 of the container main body 3 due to influence of the ambient temperature during pulverization of a sample at normal temperature can be inhibited. Thus, part of a frozen sample being pulverized can be inhibited from melting and freezing again at the inner surface of the side plate 6 to form lumps and adhere to the inner surface of the side plate 6. Accordingly, the uniformity of the pulverized sample improves and a uniformized pulverized sample can be more efficiently obtained, and also wasteful consumption of dry ice can be prevented.

Moreover, as compared to a configuration in which the periphery of the side plate of the pulverizing container is covered with a heat insulator, work to remove the heat insulator in washing the container and the heat insulator and work to mount the heat insulator after washing are unnecessary, so that the maintenance workability is enhanced.

In addition, since the filter member F, which prevents a sample within the pulverizing container 1 from passing therethrough while allowing gas to pass therethrough, is provided in the vent hole 7 formed in the center portion of the lid 4, carbon dioxide gas generated in a large amount when dry ice sublimates during dry ice freeze-pulverization is discharged through the vent hole 7 of the lid 4, the filter member F, and the through hole 8A of the filter holder 8, and also the sample does not bubble over. Thus, a uniformized pulverized sample can be still further efficiently obtained, and contamination of the surroundings can be prevented.

The configuration has been described above in which the side plate 6 and the bottom plate 5 of the container main body 3 are fowled with the vacuum double structure and the radiant heat transfer prevention bodies Q1 and Q2 are provided in the internal spaces S1 and S2, respectively. However, as shown in a main part enlarged cross-sectional front view of FIG. 4, the lid 4 may be formed with a vacuum double structure that is obtained by evacuating an internal space S3 formed by opposing metal plates P3A and P3B that are, for example, stainless steel plates, and a radiant heat transfer prevention body Q3 that serves as a mirror and is formed from, for example, copper foil may be provided on the outer surface (vacuum-side surface) of the metal plate P3B of the internal space S3.

The configuration in which the radiant heat transfer prevention bodies Q1, Q2, and Q3 are provided to the pulverizing container 1 is effective for significantly reducing heat transmission by radiation. However, depending on requirement specifications or the like, the side plate 6 and the like may merely be formed with a vacuum double structure, and the radiant heat transfer prevention bodies Q1, Q2, and Q3 may not be provided.

In addition, regarding the vacuum double structure of the container main body 3, only at least the side plate 6 needs to be formed with a vacuum double structure, and the heat insulation properties are further enhanced when the bottom plate 5 and the lid 4 are formed with a vacuum double structure.

### DESCRIPTION OF THE REFERENCE CHARACTERS

1 pulverizing container

2 pulverizer main body

3 container main body

4 lid

4A recess

5 bottom plate

6 side plate

7 vent hole

8 filter holder

8A through hole

A dry ice freeze-pulverizer

B upper surface opening

C pulverizing blade

D drive device

E operation panel

F filter member

G grip P1A, P1B opposing metal plate

P2A, P2B opposing metal plate

P3A, P3B opposing metal plate

Q1, Q2, Q3 radiant heat transfer prevention body

S1, S2, S3 internal space

1. A dry ice freeze-pulverizer comprising:
a pulverizing container including a container main body that includes a bottom plate and a side plate erected from a peripheral edge of the bottom plate, has an upper surface opening, and is provided with a pulverizing blade therein, and a lid that closes the upper surface opening and has a vent hole: and
a pulverizer main body including a drive device configured to rotate the pulverizing blade, wherein
the side plate of the container main body is formed with a vacuum double structure that is obtained by evacuating an internal space formed by opposing metal plates.

2. The dry ice freeze-pulverizer according to claim 1, wherein one or each of the bottom plate and the lid is formed with a vacuum double structure that is obtained by evacuating an internal space formed by opposing metal plates.

3. The dry ice freeze-pulverizer according to claim 1, wherein a radiant heat transfer prevention body that serves as a mirror is provided in the internal space.

4. The dry ice freeze-pulverizer according to claim 1, wherein a filter member that prevents a sample within the pulverizing container from passing therethrough while allowing gas to pass therethrough is provided in the vent hole of the lid.

5. The dry ice freeze-pulverizer according to claim 2, wherein a radiant heat transfer prevention body that serves as a mirror is provided in the internal space.

6. The dry ice freeze-pulverizer according to claim 2, wherein a filter member that prevents a sample within the pulverizing container from passing therethrough while allowing gas to pass therethrough is provided in the vent hole of the lid.

7. The dry ice freeze-pulverizer according to claim 3, wherein a filter member that prevents a sample within the pulverizing container from passing therethrough while allowing gas to pass therethrough is provided in the vent hole of the lid.

8. The dry ice freeze-pulverizer according to claim 5, wherein a filter member that prevents a sample within the pulverizing container from passing therethrough while allowing gas to pass therethrough is provided in the vent hole of the lid.
